(19) United States
(12) Patent Application Publication
(10) Pub. No.: US 2016/0113836 A1
(43) Pub. Date: Apr. 28, 2016

(54) Handheld Apparatus with Heated Roller

(71) Applicant: Brady Walker Frazier, Carrollton, TX (US)

(72) Inventor: Brady Walker Frazier, Carrollton, TX (US)

(21) Appl. No.: 14/508,286

(22) Filed: Oct. 24, 2014

(51) Int. Cl. A61H 15/00; A61F 7/00

(52) U.S. Cl. CPC A61H 15/0092; A61F 7/00; A61H 2015/0007; A61F 2007/0087

(57) ABSTRACT

An apparatus is disclosed for applying heat and pressure to a contusion. The disclosed apparatus comprises a housing, at least one roller, and a heating element. The heating element is configured to heat the roller, and the roller is configured to be applied to a contusion.

### TECHNICAL FIELD

The present invention relates generally to a handheld apparatus with a heated roller.

### BACKGROUND

When soft tissue is injured, blood vessels may be broken, leaving a hematoma of the tissue known as a contusion. Generally, broken blood vessels occur in the upper layers of skin and surrounding tissue. A contusion initially presents itself as black or blue area under the skin, the size of which may vary based on the nature and severity of the injury. The discoloration is caused by blood that has leaked out of the broken blood vessels and into the surrounding interstitial tissue. After the blood coagulates, the contusion begins to fade as the blood is reabsorbed into the body through the process of phagocytosis. During this process, the contusion may get lighter in color, fading from blue or purple to green and yellow. The process of the contusion fading from sight may take several days or even weeks depending on the nature of the injury and the speed of the body's healing ability. The tissue damage that caused the contusion may finish healing long before the contusion finishes fading, leaving an unaesthetic discoloration that may last for days.

It is well-known that a cold compress may aid in the prevention of contusion formation if applied soon after a contusion-causing injury occurs. The cold compress acts to lower the temperature of the area to which it is applied and thereby slow down blood flow and contract the blood vessels around the injured area. However, there is currently no popular, or particularly effective, method or product that is used to treat the aesthetic appearance of contusions after they have begun to heal. As such, it may be desirable to configure an apparatus for improving the aesthetic appearance of a contusion.

### SUMMARY OF THE INVENTION

Various aspects of examples of the present invention are set out in the claims.

One or more embodiments may provide an apparatus comprising a housing, wherein the housing comprises a head and a body; at least one roller, wherein the roller is configured to rotate within the head; and a heating element, wherein said heating element is configured to heat the roller.

One or more embodiments may provide an apparatus comprising a housing, wherein the housing comprises a head and a body; at least one roller; a pin, wherein the roller is mounted to the pin, wherein the pin is rotatably mounted to the head; and a heating element, wherein the heating element is mounted to the pin, wherein the heating element is configured to heat the roller; a power supply, wherein the power supply is configured to supply electrical current to the heating element; and a switch, wherein the switch is configured to control the electrical output of the power supply.

One or more embodiments may provide a method comprising providing an apparatus, wherein the apparatus comprises a housing comprising a head and a body, at least one roller, wherein the roller is configured to rotate within the head, and a heating element, wherein said heating element is configured to heat the roller above room temperature; and rolling the apparatus on the roller against a surface of the skin proximate to a contusion.

Additional benefits and advantages of the present invention will become apparent to those skilled in the art to which it pertains upon a reading and understanding of the following detailed specification.

### BRIEF DESCRIPTION OF THE DRAWINGS

The foregoing and other advantages and features of the invention will become apparent upon reading the following detailed description with reference to the accompanying drawings in which:

FIG. 1 is a side view of the apparatus of the invention according to at least one example embodiment.

FIG. 2 is an axonometric view of the apparatus of FIG. 1.

FIG. 3 is a top view of the apparatus of FIG. 1.

FIG. 4 is an axonometric view of the roller of the apparatus according to at least one example embodiment.

### DETAILED DESCRIPTION OF THE INVENTION

In at least one example embodiment, the apparatus of the invention comprises a housing. The housing may be made out of a polymeric material such as plastic, a metal such as stainless steel or aluminum, and/or the like. The housing may comprise a body portion configured to be held in the user's hand. The body may additionally comprise a rubber grip along its surface.

The housing may further comprise a head portion configured to retain at least one roller. The head may be substantially solid, substantially hollow, and/or the like. Furthermore, it may be desirable to configure the head in such way that the head is removable from the housing via a lock, latch, screw cap, and or the like. In this manner, the head may be easily replaced or substituted.

The roller or plurality of rollers may comprise an elastomeric material such as polybutadiene rubber. However, the roller may also comprise any suitable material such as plastic, metal, silicone gel, and/or the like. It may be desirable to configure the shape of the roller according to a number of different shapes, including but not limited to a cylinder, a tapered cylinder, a sphere, and/or the like, in order to suit the preferences of the user. Similarly, the roller may also comprise massaging ridges, bumps, and/or the like. Furthermore, it may be desirable to configure the roller in such a way that it may be removed from or added to the head portion without requiring removal the head, or otherwise requiring disassembly of the housing. Such a configuration may allow the user to easily substitute a variety of different roller shapes based, at least in part, on the user's preferences, the nature of the contusion, and/or the like.

The roller may comprise a pin which runs through the length of the roller and connects to the head of the apparatus exterior to the roller. It may be desirable for the pin to comprise a heating element. For example, such a configuration may allow the heating element to increase the temperature of the roller to within a temperature range. Furthermore, the roller may feature holes that act as heat channels for the heat output by the heating element. The heating element may comprise an electrical resistor that creates heat by means of resistance to an electric current from a power supply. However, the heating element may be any form of heating device configured to properly heat the roller. The electric current may be supplied to the heating element by conductive wires. The wires may be located interior to the housing.

The housing may further comprise a power supply cover that covers a power supply cavity. The power supply cover may be configured to be opened by a tab and swing out from the housing on hinges. The tab may comprise a detent of the sort that is well known in the art, said detent allowing the tab to latch to the housing and remain closed unless acted upon by a predetermined amount of force perpendicular to the surface of the power supply cover. The housing may be configured to receive a power supply within the power supply cavity. The power supply is may be standard batteries, but may also be any other suitable type of battery or alternative power supply such as rechargeable batteries, an electrical cord, or a wireless charger.

The power supply cavity and power supply cover may be located on the side or bottom surface of the housing. In this manner, they may be out of the way of the user while the apparatus in use. However, the power supply cavity and power supply cover may be located at an alternate location on the housing, or may take an entirely different form such as a screw cap. In some circumstances, it may be desirable for the apparatus to use wireless charging, an electrical cord, and/or the like. In circumstances such as these, the apparatus may be configured such that there is no power supply cover or power supply cavity, or further configured such that the power supply is only accessible by taking the apparatus apart via fasteners that may hold the housing together.

The housing may further comprise a switch for turning the apparatus on. In this context, turning the apparatus on may mean providing power to a heating element interior to a roller, thereby heating the roller. The switch may have two settings, ON and OFF, which may be marked on the exterior of the housing. The ON setting may automatically heat the heating element to within an optimal temperature range. For example, an optimal, temperature range may include temperatures generally above 100° Fahrenheit and generally below 140° Fahrenheit. Alternately, the switch may also comprise a range of possible positions which relate to different temperature ranges at which the heating element may be set. The switch may be located near the center of the upper surface of the housing. In this manner, the switch may be easily accessed by the thumb of a user. However, the switch may also be located on any other suitable portion of the housing and may take any workable form. For example, the switch may take the form of a rotary dial, an electronic touchpad, a mechanical button, and/or the like.

In circumstances where a roller reaches a desired temperature, the roller may be applied to a contusion by rolling the apparatus against the surface of the skin proximate to the contusion. The heat and pressure from the roller may help to dissipate coagulated blood within the contusion. In this manner, the coagulate blood may spread out under the skin so that the contusion may be less noticeable upon visual inspection and may be reabsorbed by the body more quickly. The apparatus may be applied to a contusion for a length of time based, at least it part, on the preference of the user. A number of applications over a period of days may be necessary in order for the desired aesthetic improvements to be achieved.

It may be desirable to configure the pin such that the pin is attached to springs at the ends of the pin, wherein the springs bias the roller against the surface of the skin. In this manner, the roller may be depressed inward in circumstances where sufficient force is applied against the skin by the user. This configuration may redirect some portion of the user's force into the springs. The user of the applicator may thereby be constrained from using more force against the contusion than is advisable. The material and dimensions of the springs may control the amount of force that is necessary to compress the springs a certain distance. The material and dimensions of the springs may be varied based, at least in part, on an optimal treatment plan for the contusion.

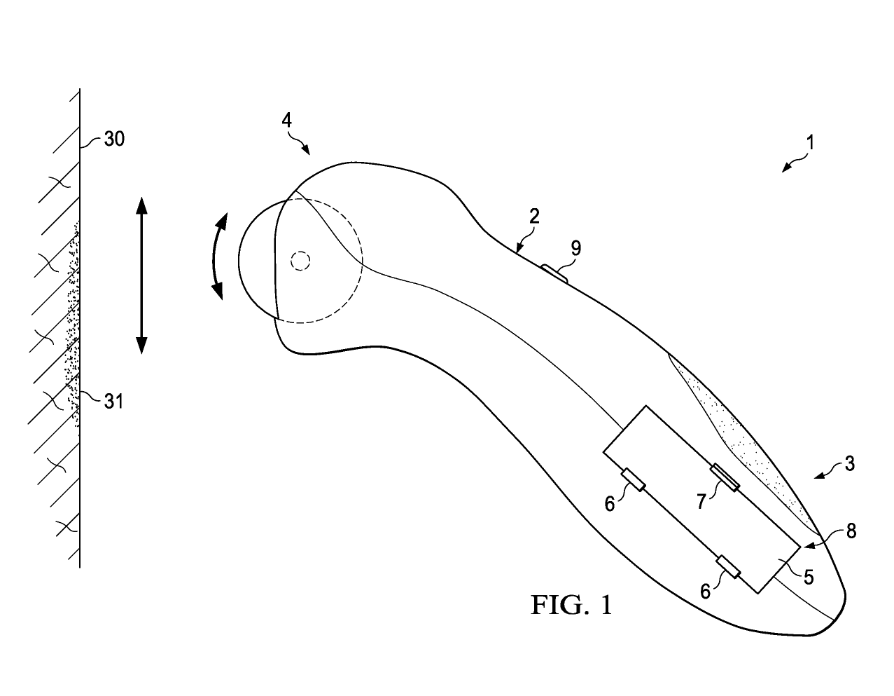

FIGS. 1-4 are diagrams illustrating an apparatus of the invention according to at least one example embodiment. The apparatus of the invention may be used in a variety of applications and under a variety of conditions. The examples of FIGS, 1-4 are merely examples of an apparatus of the invention and do not limit the scope of the claims.

FIG. 1 illustrates one embodiment of the apparatus 1 of the invention. As shown in FIG. 1, apparatus 1 comprises a housing 2, where the housing 2 features a body 3 designed for handheld use and a head 4 designed to retain a roller 20. As shown, the body 3 features a switch 9 that controls that power output from the power supply. The power supply is contained within the power supply cavity 8, which is covered by the power supply cover 5. The power supply cover 5 comprises a tab 7 and one or more hinges 6. When the user needs to access the power supply cavity 8, for instance to change a depleted power supply with a new or recharged power supply, the user may lift the tab 7 of the power supply cover 5, causing the power supply cover 5 to swing open on the one or more hinges 6.

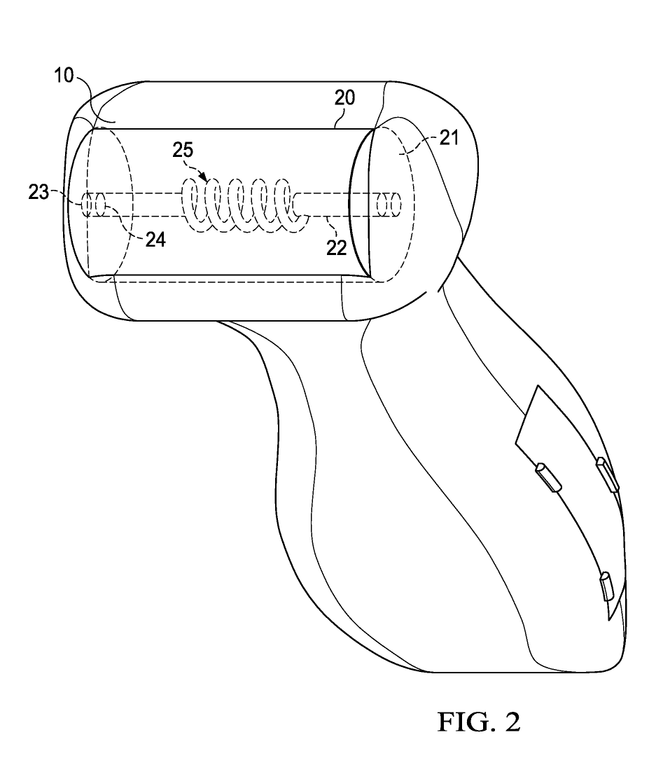

The roller 20 depicted in FIG. 2 is located in the head 4 of the apparatus 1. The roller 20 comprises a pin 22 that runs through the transverse axis of the roller 20 and exits out of the roller 20 at the pin exit points 24. The pin 22 is held in place within the head 4 at the pin ends 23. The pin ends 23 are mounted to the head 4 in such a fashion that the roller 20 is free to rotate about its transverse axis.

FIG. 2 further depicts the pin 22 comprising a heating element 25 located along the length of the pin 22 interior to the roller 20. Generally, the heating element 25 is designed to heat up in response to resistance from the electrical current supplied by the power supply. The heat from the heating element 25 penetrates into the roller 20, causing the temperature of the roller 20 to be elevated.

As FIGS. 1 and 2 illustrate, in use the apparatus 1 may be turned ON via the switch 9 such that the heating element 25 begins to heat up the roller 20. Once the roller 20 reaches the desired temperature, the user rolls the apparatus 1 against the surface 30 of the skin proximate to a contusion 31. As the heated roller 20 makes repeated rolling contact with the contusion 31, the pressure, as generated by the user, and heat from the roller 20 act to increase the temperature and mobility of the coagulated blood at the site of the contusion, allowing the blood to spread out under the surface 30 of the skin such that it appears lighter in color. Once the user is finished using the apparatus 1, the apparatus 1 may be turned OFF via the switch 9 and stored for future use.

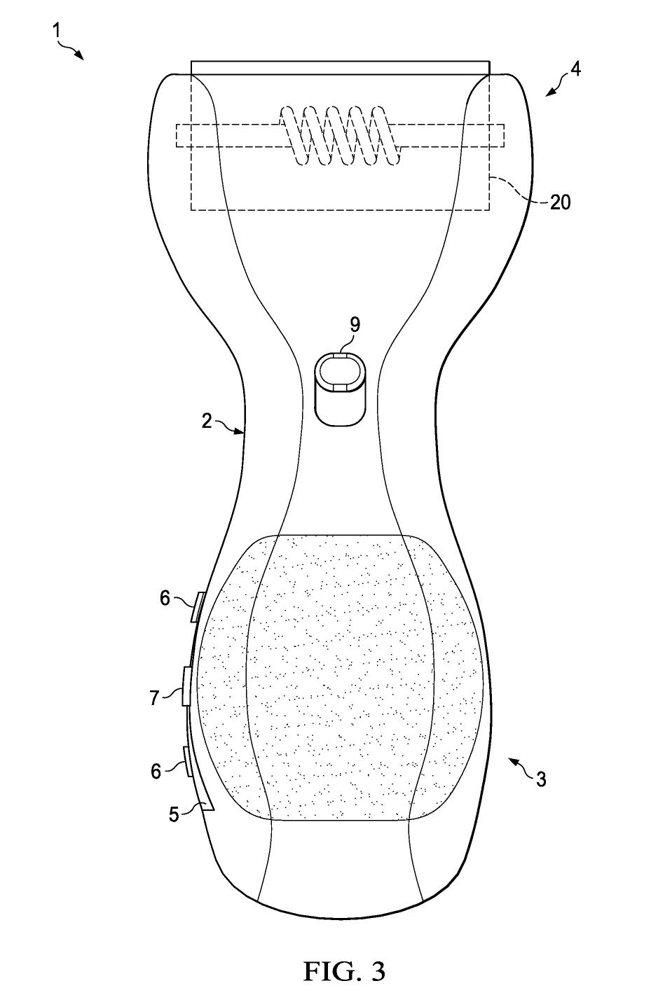

FIG. 3 illustrates a top view of the embodiment of the apparatus 1 from FIG. 1, wherein the switch 9 is generally located proximate to the center of the upper side of the housing 2 so that it may be easily accessed by the thumb of the user while being held. Furthermore, FIG. 3 depicts the power supply cover 5 being located on the left side of the body 3 so as to offer the least contact possible with a right-handed user of the apparatus 1. However, both the switch 9 and the power supply cover 5 could alternately be located elsewhere on the housing 2 so as to maximize compatibility with alternate hand geometries or user preferences.

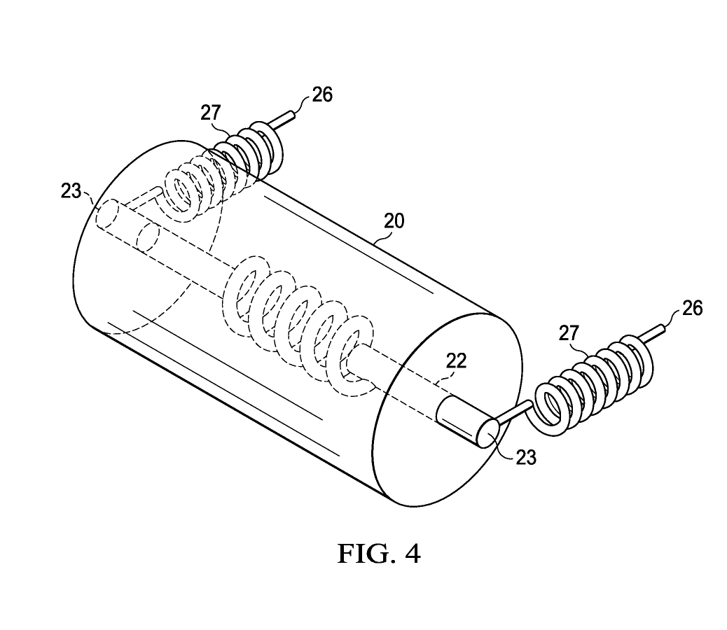

FIG. 4 illustrates an alternate embodiment of the apparatus 1. As shown in FIG. 4, the roller 20 may be configured to depress into the head 4 of the housing 2 when it is pushed against the surface 30 of the skin with sufficient force. In this alternate embodiment, the pin ends 23 are not held in place by the head 4, but rather are rotatably attached to springs 27 that are biased against the surface 30. The springs are attached to the head 4 at hard points 26 interior to the surface of the housing 2. When the roller 20 is pushed against the surface 30 by the user, the springs 27 may compress in the direction of the hard points 26. As the springs 27 compress, the roller 20 depresses into the head 4 and potential energy generated by the user is stored in the springs 27 until the user decreases the amount of force exerted by the apparatus 1 against the surface 30. In so doing, the springs 27 help safeguard the user from exerting too much force on the contusion 31 by making it more difficult to do so.

Although not depicted in FIGS. 1-4, it will be understood by one of ordinary skill in the art that the head 4 may be configured to retain a plurality of rollers 20. Furthermore, it will be understood by one of ordinary skill in the art that a user of the invention may find cause to apply the apparatus 1 to a surface 30 of the skin that is not proximate to a contusion 31, such as a surface 30 proximate to a sore muscle.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the above describes example embodiments of the invention, these descriptions should not be viewed in a limiting sense. Rather, there are variations and modifications which may be made without departing from the scope of the present invention as defined in the appended claims.

What is claimed is:

1. An apparatus, said apparatus comprising:
a housing, wherein the housing comprises a head and a body;
at least one roller, wherein the roller is configured to rotate within the head; and
a heating element, wherein the heating element is configured to heat the roller.

2. The apparatus according to claim 1, wherein the roller is mounted on a pin.

3. The apparatus according to claim 2, wherein the pin further comprises one or more pin ends, wherein the one or more pin ends are rotatably mounted to the head.

4. The apparatus according to claim 2, wherein the heating element is mounted to the pin.

5. The apparatus according to claim 4, wherein the heating element is an electrical resistor.

6. The apparatus according to claim 1, wherein the apparatus further comprises a power supply, wherein the power supply is electrically connected to the heating element.

7. The apparatus according to claim 6, wherein the apparatus further comprises a power supply cavity, wherein the power supply may be secured within the power supply cavity by a power supply cover.

8. The apparatus according to claim 7, wherein the power supply cover comprises a latch and one or more hinges, wherein the power supply cover is rotatably mounted to the one or more hinges.

9. The apparatus according to claim 6, wherein the apparatus further comprises a switch, wherein the switch controls the electrical output of the power supply.

10. The apparatus according to claim 9, wherein the switch has two or more positions, wherein each position relates to a different level of electrical output of the power supply.

11. The apparatus according to claim 3, wherein the head further comprises one or more springs, wherein the one or more springs are mounted to the head at one or more hard points, wherein the one or more pin ends are rotatably mounted to the one or more springs.

12. An apparatus, said apparatus comprising:
a housing, wherein the housing comprises a head and a body;
at least one roller;
a pin, wherein the roller is mounted on the pin, wherein the pin is rotatably mounted to the head;
a heating element, wherein the heating element is mounted to the pin, wherein the heating element is configured to heat the roller;
a power supply, wherein the power supply is configured to supply electrical current to the heating element; and
a switch, wherein the switch is configured to control the electrical output of the power supply.

13. A method, said method comprising:
providing an apparatus, said apparatus comprising:
a housing, wherein the housing comprises a head and a body;
at least one roller, wherein the roller is configured to rotate within the head;
and a heating element, wherein the heating element is configured to heat the roller; and
rolling the roller of the apparatus against a surface of skin proximate to a contusion.

14. The method according to claim 13, wherein the roller is mounted on a pin.

15. The method according to claim 14, wherein the pin further comprises one or more pin ends, wherein the one or more pin ends are rotatably mounted to the head.

16. The method according to claim 14, wherein the heating element is mounted to the pin, wherein the heating element is an electrical resistor.

17. The method according to claim 13, wherein the apparatus further comprises a power supply, wherein the power supply is electrically connected to the heating element.

18. The method according to claim 17, wherein the apparatus further comprises a power supply cavity, wherein the power supply may be secured within the power supply cavity by a power supply cover.

19. The method according to claim 13, wherein the apparatus further comprises a switch, wherein the switch controls the electrical output of the power supply.

20. The method according to claim 19, wherein the switch has two or more positions, wherein each position relates to a different level of electrical output of the power supply.
